(19) United States
(12) Patent Application Publication
(10) Pub. No.: US 2015/0081028 A1
(43) Pub. Date: Mar. 19, 2015

(54) ADAPTOR FOR MODULAR JOINT PROSTHESES

(71) Applicants: Ray Zubok, Midland Park, NJ (US); Keith A. Roby, Jersey City, NJ (US); Timothy A. Hoeman, Morris Plains, NJ (US); Imants Liepins, Asbury, NJ (US); John Chernosky, Brick, NJ (US)

(72) Inventors: Ray Zubok, Midland Park, NJ (US); Keith A. Roby, Jersey City, NJ (US); Timothy A. Hoeman, Morris Plains, NJ (US); Imants Liepins, Asbury, NJ (US); John Chernosky, Brick, NJ (US)

(21) Appl. No.: 14/489,819

(22) Filed: Sep. 18, 2014

Related U.S. Application Data
(60) Provisional application No. 61/879,435, filed on Sep. 18, 2013.

(51) Int. Cl. A61F 2/38

(52) U.S. Cl. CPC A61F 2/3854
USPC 623/20.15; 623/20.24

(57) ABSTRACT

A bone augment adaptor is configured to be inseparably coupled to a bone augment. A first end of the adaptor is configured to be connected to an epiphyseal replacement portion of a modular joint replacement prosthesis. A second end of the adaptor may be configured to be connected to a diaphyseal anchoring portion of the prosthesis.

### CLAIM OF PRIORITY

This application claims the benefit of U.S. Provisional Patent Application Ser. No. 61/879,435, filed on Sep. 18, 2013, the benefit of priority of which is claimed hereby, and which is incorporated by reference herein in its entirety.

### BACKGROUND

Joint replacement procedures are commonly performed to alleviate pain and loss of function in injured and diseased joints. A human knee is a joint, for example, connects a femur to a tibia (sometimes referred to as the thigh bone and the shin bone, respectively). The knee allows for pivoting between the femur and the tibia. The pivoting has a pivot axis aligned with the medial-lateral direction. Some types of injury, disease, or degeneration can produce pain and/or restricted motion in the knee joint. One treatment for certain types of damage to a knee joint is surgery. For relatively mild knee damage, the knee may be repaired. For more severe damage, the knee may be replaced.

In total knee replacement surgery, all of the articulating elements within the knee joint are replaced. During the surgery, a distal end (sometimes referred to as an inferior end or a bottom end) of the femur is cut to a particular shape, and then a femoral implant is attached to the cut distal end of the femur. The femoral implant typically includes a pair of convex condylar surfaces. The condylar surfaces are shaped to slide within corresponding concave bearing indentations on a tibial bearing surface. The tibial bearing surface is typically formed from a hard plastic, which allows the condylar surfaces to slide in the indentations with reduced friction.

In some surgical cases, there has been a loss of bone at the distal portion of the femur and/or the proximal portion of the tibia. In order to compensate for the missing bone, a bone augments can be employed along with the other components of the prosthesis. The augments can be attached between an epiphyseal replacement portion (e.g. the articulating portion) and a diaphyseal anchoring portion (e.g. the stem) of the joint replacement prosthesis.

Modular prosthetic components are useful, at least in part, because they allow the surgeon to assemble components in a variety of configurations at the time of surgery to meet specific patient needs relative to size and geometry. For example, modular femoral components can include separate stem and articulating condylar components that can be assembled in a variety of configurations. Likewise, modular tibial components can include separate convex tibial bearing components, tibial platforms, and stems, which can be assembled in a variety of configurations. The use of augments in prosthetic systems can complicate modularity, as surgeons may wish to mix and match augments and femoral and tibial prosthetic components from different surgical kits and from different manufacturers.

### SUMMARY

Examples according to this disclosure are directed to an adaptor that can be inseparably coupled to a bone augment and which is configured to be connected, at one end of the adaptor, to an epiphyseal replacement portion and, in some cases, is configured to be connected, at the other end of the adaptor, to a diaphyseal anchoring portion of a modular joint replacement prosthesis.

Modular joint replacement prostheses can increase the adaptability of a prosthesis system to varying degrees of damage and/or disease to the joint, which may, in turn, improve surgical outcomes. Modular joint replacement systems generally include a prosthesis for each bone of the joint. Each prosthesis can include separate epiphyseal replacement portions and diaphyseal anchoring portions. Example epiphyseal replacement portions include a proximal femoral component in a hip prosthesis, a distal femoral component in a knee prosthesis, a proximal tibial component in a knee prosthesis, and a distal humeral component in a shoulder prosthesis. The diaphyseal anchoring portions can include an intramedullary stem configured to be implanted within a medullary cavity of the diaphysis of a bone of the joint. The epiphyseal portions can include multiple components or sections. For example, a proximal femoral component can include a neck and/or body interposed between the intramedullary stem and a femoral head.

Joint replacement prosthesis may also include bone augments, which are configured to compensate for bone loss that occurs as the result of damage and/or disease to the joint and/or as the result of the surgical procedure to replace or repair the joint. Bone augments can be adapted for different portions of a bone. For example, joint prostheses can include one or both of metaphyseal and diaphyseal augments. In any case, the augments are generally arranged between the epiphyseal replacement portions and diaphyseal anchoring portions of a modular joint replacement prosthesis.

The use of augments in prosthetic systems can complicate modularity, as surgeons may wish to mix and match augments and femoral and tibial prosthetic components from different surgical kits and from different manufacturers. As such, examples according to this disclosure are directed to an adaptor that can be inseparably coupled to a bone augment and which is configured to be connected to an epiphyseal replacement portion and to a diaphyseal anchoring portion of a modular joint replacement prosthesis.

One example according to this disclosure includes an adaptor for a modular prosthetic device configured to partially or completely replace a human joint. The adaptor includes a tapered outer surface and first and second ends. The tapered outer surface is configured to be received within a cavity of an augment. The cavity includes a tapered inner surface. The tapered outer surface of the adaptor and the tapered inner surface of the central cavity of the augment are configured to interlock the adaptor and the augment. The first end of the adaptor is configured to be coupled to an epiphyseal component of the modular prosthetic device. The second end of the adaptor is configured to be coupled to an intramedullary stem of the modular prosthetic device.

In other examples, the augment and the adaptor can be coupled to one another by mechanisms other than interlocking tapered surfaces. For example, the augment and adaptor can be press or interference fit to one another. Regardless of how the two components are connected, the augment and adaptor are configured to be generally inseparable and the adaptor is configured to be connected, at one end, to an epiphyseal replacement portion and, at the other end, to a diaphyseal anchoring portion of a modular joint replacement prosthesis. In one example, the augment and adaptor are inseparably coupled to one another and the adaptor is configured to be connected, at one end, to a number of different epiphyseal replacement portions and, at the other end, to a number of different diaphyseal anchoring portions.

The details of examples of the disclosure are set forth in the accompanying drawings and the description below. Other features, objects, and advantages of examples according to this disclosure will be apparent from the description and drawings, and from the claims.

### BRIEF DESCRIPTION OF DRAWINGS

FIG. 1 schematically depicts an example modular knee repair or replacement prosthesis including a bone augment adaptor in accordance with this disclosure.

FIGS. 2A and 2B depict elevation and section views, respectively, of a distal femoral prosthesis including an example adaptor in accordance with this disclosure.

FIG. 3 depicts an example proximal tibial prosthesis including another example adaptor in accordance with this disclosure.

FIGS. 4A and 4B depict the adaptor, bone augment, and bushing of the proximal tibial prosthesis of FIG. 3 in greater detail.

FIG. 5 is a flowchart depicting an example method in accordance with this disclosure.

FIG. 6 depicts a section view of a distal femoral prosthesis including another example adaptor in accordance with this disclosure.

FIG. 7 depicts a section view of another example adaptor in accordance with this disclosure.

FIG. 8 schematically depicts an example modular knee repair or replacement prosthesis including adaptors in accordance with this disclosure.

### DETAILED DESCRIPTION

As noted above, examples according to this disclosure are directed to adaptors that can be inseparably coupled to a bone augment and which are configured to be connected, at one end of the adaptor, to an epiphyseal replacement portion and, in some cases, at the other end of the adaptor, to a diaphyseal anchoring portion of a modular joint replacement prosthesis.

One example of a joint prosthesis is a knee repair or replacement prosthesis, which can include a femoral prosthesis and/or a tibial prosthesis. The femoral prosthesis can include an epiphyseal replacement portion including a pair of convex condylar surfaces that can slide within corresponding concave bearing indentations on a tibial bearing surface of the tibial prosthesis. The condylar component of the femoral prosthesis can be coupled to an intramedullary stem, which extends proximally from the condylar component, and attaches to a cut distal end of the femur. The tibial bearing surface of the tibial prosthesis can be disposed on a proximal side of a tibial platform. An intramedullary stem extends distally from the tibial platform, and attaches to a cut proximal end of the tibia.

One example according to this disclosure includes an adaptor for such a knee prosthesis. The adaptor includes a tapered outer surface and first and second ends. The tapered outer surface of the adaptor is configured to be received within a cavity of a bone augment. The bone augment could be, e.g., a diaphyseal or metaphyseal femoral augment that augments the cut proximal end of the femur to which the prosthesis is attached. The cavity of the augment includes a tapered inner surface. For example, the femoral augment can include a central bore that includes a tapered profile along a portion or all of the axial length of the bore. The tapered outer surface of the adaptor and the tapered inner surface of the central cavity of the augment are configured to interlock the adaptor and the augment.

The first end of the adaptor is configured to be coupled to an epiphyseal component of the knee repair or replacement prosthesis. For example, the first end of the adaptor can be coupled to a distal femoral component, which includes medial and lateral condyles. The second end of the adaptor is configured to be coupled to an intramedullary stem of the knee repair or replacement prosthesis, which is configured to be affixed within the medullary cavity of the femur.

As used herein, “proximal” refers to a direction generally toward the torso of a patient, and “distal” refers to the opposite direction of proximal, i.e., away from the torso of a patient. “Anterior” refers to a direction generally toward the front of a patient or knee, and “posterior” refers to the opposite direction of anterior, i.e., toward the back of the patient or knee. In the context of a prosthesis alone, such directions correspond to the orientation of the prosthesis after implantation, such that a proximal portion of the prosthesis is that portion which will ordinarily be closest to the torso of the patient, the anterior portion closest to the front of the patient's knee, etc.

Similarly, knee and other prostheses and augments in accordance with the present disclosure may be referred to in the context of a prosthesis coordinate system including three mutually perpendicular reference planes, referred to herein as the transverse, coronal and sagittal planes of the knee prosthesis. Upon implantation and with a patient in a standing position, a transverse plane of the knee prosthesis is generally parallel to an anatomic transverse plane, i.e., the transverse plane is inclusive of imaginary vectors extending along medial/lateral and anterior/posterior directions. Coronal and sagittal planes of the knee prosthesis are also generally parallel to the coronal and sagittal anatomic planes in a similar fashion. Thus, a coronal plane of the prosthesis is inclusive of vectors extending along proximal/distal and medial/lateral directions, and a sagittal plane is inclusive of vectors extending along anterior/posterior and proximal/distal directions. As with anatomic planes, the sagittal, coronal and transverse planes of a knee prosthesis are mutually perpendicular to one another. For purposes of the present disclosure, reference to sagittal, coronal and transverse planes is with respect to a knee prosthesis unless otherwise specified.

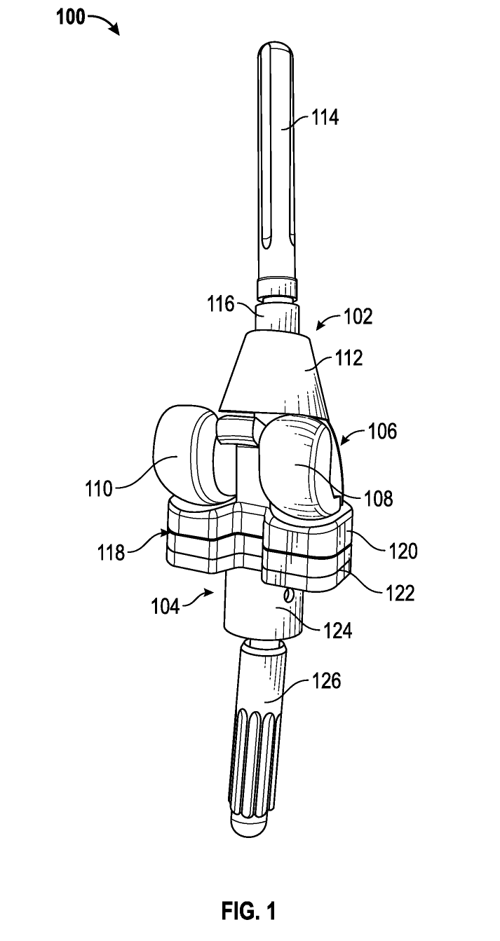

FIG. 1 schematically depicts example modular knee repair or replacement prosthesis 100, which includes bone augment adaptors in accordance with this disclosure. In FIG. 1, knee prosthesis 100 includes distal femoral prosthesis 102 and proximal tibial prosthesis 104. Distal femoral prosthesis 102 includes epiphyseal replacement portion 106 including medial and lateral condyles 108, 110, respectively. Distal femoral prosthesis 102 also includes diaphyseal bone augment 112, intramedullary stem 114, and adaptor 116. Diaphyseal augment 112 is interposed between epiphyseal replacement portion 106 and intramedullary stem 114. Adaptor 116 is arranged within and coupled to a central cavity of augment 112. The proximal end of adaptor 116 is connected to intramedullary stem 114 and the distal end is connected to epiphyseal replacement portion 116.

Proximal tibial prosthesis 104 includes epiphyseal replacement portion 118 including tibial bearing 120 and platform 122. Tibial prosthesis 104 also includes augment 124 and intramedullary stem 126. The distal side of tibial bearing 120 is connected to the proximal side of platform 122. Although not shown in FIG. 1, platform 122 can include a protrusion extending distally into a central cavity of augment 124. Bone augment 124 is interposed between platform 122 and intramedullary stem 126. Intramedullary stem 126 can extend through the central cavity of augment 124 to connect to platform 122.

Portions or all of distal femoral prosthesis 102 and proximal tibial prosthesis 104 can be fabricated from a variety of biologically compatible materials and by a variety of processes including machining, casting, forging, compression molding, injection molding, sintering, and/or other suitable processes. In some examples, all of the portions of femoral prosthesis 102 and/or tibial prosthesis 104 are fabricated from the same material, while, in other examples, different portions of the prostheses are fabricated from different materials. In one example, one or more portions of femoral prosthesis 102 and/or tibial prosthesis 104 are fabricated from metals, polymers, ceramics, and/or other suitable materials. For example, one or more portions of femoral prosthesis 102, including adaptor 116, and/or tibial prosthesis 104 may be made of a cobalt-chromium alloy. Other metals suitable for femoral prosthesis 102, including adaptor 116, and/or tibial prosthesis 104 (including in combination with cobalt and/or chrome) include titanium, aluminum, vanadium, molybdenum, hafnium, nitinol, molybdenum, tungsten, nickel, tantalum, and stainless steel.

The example of FIGS. 1 is described with reference to adaptor 112 in accordance with this disclosure included in distal femoral prosthesis 102. However, in other examples, an adaptor in accordance with this disclosure could also be included in a modular tibial prosthesis like proximal tibial prosthesis 104. For example, tibial prosthesis 104 could include an adaptor that is arranged within and coupled to the central cavity of augment 124. In such an example, proximal end of the tibial adaptor could be connected to intramedullary stem 126 and the distal end could be connected to epiphyseal replacement portion 118.

In practice, knee prosthesis 100 is configured to be implanted in a patient to alleviate damage and/or disease of the patient's knee. Distal femoral prosthesis 102 is implanted in the distal end of a femur of the patient and replaces the epiphyseal portion of the femur, including the articulating medial and lateral condyles. Tibial prosthesis 104 is implanted in the proximal end of a tibia of the patient and replaces the epiphyseal portion of the tibia, including the articular surfaces of the tibia bearing the condyles.

Medial and lateral condyles 108, 110 of epiphyseal replacement portion 116 each include convex bearing surfaces that are configured to approximate the condyles of the patient's femur. Tibial bearing 120 includes concave bearing surfaces with which the convex surfaces of condyles 108 and 110 are configured to articulate.

Intramedullary stem 114 anchors distal femoral prosthesis 102 to the patient's femur by being affixed to the medullary cavity of the femur. Similarly, intramedullary stem 126 anchors tibial prosthesis 104 to the patient's tibia by being affixed to the medullary cavity of the tibia.

Diaphyseal femoral bone augment 112 of distal femoral prosthesis 102 and tibial bone augment 124 of tibial prosthesis 104 are configured to compensate for bone loss that occurs as the result of damage and/or disease to the joint and/or as the result of the surgical procedure to replace or repair the joint. Bone augments can be adapted for different portions of a bone. For example, although not illustrated in FIG. 1, distal femoral prosthesis 102 can include a metaphyseal bone augment in addition to diaphyseal augment 112. Such a metaphyseal bone augment could be arranged between diaphyseal augment 112 and medial and lateral condyles 108, 110, respectively.

Bone augments 112 and 124 can be made of a porous bone ingrowth material that provides a scaffold for bone ingrowth on multiple surfaces. In some cases, the surfaces of augments 112 and 124 present large, three-dimensional areas of bone ingrowth material to the surrounding healthy bone for long-term fixation of the augment to the bone of the joint.

Augments 112 and 124 can be formed from one or multiple pieces of highly porous biomaterial. A highly porous metal structure can incorporate one or more of a variety of biocompatible metals. Such structures are particularly suited for contacting bone and soft tissue, and in this regard, can be useful as a bone substitute and as cell and tissue receptive material, for example, by allowing tissue to grow into the porous structure over time to enhance fixation (i.e., osseointegration) between the structure and surrounding bodily structures. In some examples, an open porous metal structure may have a porosity as low as 55%, 65%, or 75% or as high as 80%, 85%, or 90%, or within any range defined between any pair of the foregoing values. An example of an open porous metal structure is produced using Trabecular Metal™ Technology available from Zimmer, Inc., of Warsaw, Ind. Trabecular Metal™ is a trademark of Zimmer, Inc. Such a material may be formed from a reticulated vitreous carbon foam substrate which is infiltrated and coated with a biocompatible metal, such as tantalum, by a chemical vapor deposition (“CVD”) process in the manner disclosed in detail in U.S. Pat. No. 5,282,861 and in Levine, B. R., et al., “Experimental and Clinical Performance of Porous Tantalum in Orthopedic Surgery”, Biomaterials 27 (2006) 4671-4681, the disclosures of which are expressly incorporated herein by reference. In addition to tantalum, other biocompatible metals may also be used in the formation of a highly porous metal structure such as titanium, a titanium alloy, cobalt chromium, cobalt chromium molybdenum, tantalum, a tantalum alloy, niobium, or alloys of tantalum and niobium with one another or with other metals. It is also within the scope of the present disclosure for a porous metal structure to be in the form of a fiber metal pad or a sintered metal layer, such as a Cancellous-Structured Titanium™ (CSTi™) layer. CSTi™ porous layers are manufactured by Zimmer, Inc., of Warsaw, Ind. Cancellous-Structured Titanium™ and CSTi™ are trademarks of Zimmer, Inc.

Generally, a highly porous metal structure will include a large plurality of metallic ligaments defining open voids (i.e., pores) or channels therebetween. The open spaces between the ligaments form a matrix of continuous channels having few or no dead ends, such that growth of soft tissue and/or bone through open porous metal is substantially uninhibited. Thus, the open porous metal may provide a lightweight, strong porous structure which is substantially uniform and consistent in composition, and provides a matrix (e.g., closely resembling the structure of natural cancellous bone) into which soft tissue and bone may grow to provide fixation of the implant to surrounding bodily structures. According to some aspects of the present disclosure, exterior surfaces of an open porous metal structure can feature terminating ends of the above-described ligaments. Such terminating ends can be referred to as struts, and they can generate a high coefficient of friction along an exposed porous metal surface. Such features can impart an enhanced affixation ability to an exposed porous metal surface for adhering to bone and soft tissue. Also, when such highly porous metal structures are coupled to an underlying substrate, a small percentage of the substrate may be in direct contact with the ligaments of the highly porous structure, for example, approximately 15%, 20%, or 25%, of the surface area of the substrate may be in direct contact with the ligaments of the highly porous structure.

An open porous metal structure may also be fabricated such that it comprises a variety of densities in order to selectively tailor the structure for particular orthopedic applications. In particular, as discussed in the above-incorporated U.S. Pat. No. 5,282,861, an open porous metal structure may be fabricated to virtually any desired density, porosity, and pore size (e.g., pore diameter), and can thus be matched with the surrounding natural tissue in order to provide an improved matrix for tissue ingrowth and mineralization. In some examples, an open porous metal structure may be fabricated to have a substantially uniform porosity, density, and/or void (pore) size throughout, or to comprise at least one of pore size, porosity, and/or density being varied within the structure. For example, an open porous metal structure may have a different pore size and/or porosity at different regions, layers, and surfaces of the structure. The ability to selectively tailor the structural properties of the open porous metal, for example, enables tailoring of the structure for distributing stress loads throughout the surrounding tissue and promoting specific tissue ingrown within the open porous metal.

In other examples, an open porous metal structure may comprise an open cell polyurethane foam substrate coated with Ti-6Al-4V alloy using a low temperature arc vapor deposition process. Ti-6Al-4V beads may then be sintered to the surface of the Ti-6Al-4V-coated polyurethane foam substrate. Additionally, another example of an open porous metal structure may comprise a metal substrate combined with a Ti-6Al-4V powder and a ceramic material, which is sintered under heat and pressure. The ceramic particles may thereafter be removed leaving voids, or pores, in the substrate. An open porous metal structure may also comprise a Ti-6Al-4V powder which has been suspended in a liquid and infiltrated and coated on the surface of a polyurethane substrate. The Ti-6Al-4V coating may then be sintered to form a porous metal structure mimicking the polyurethane foam substrate. Further, another example of an open porous metal structure may comprise a porous metal substrate having particles, comprising altered geometries, which are sintered to a plurality of outer layers of the metal substrate. Additionally, an open porous metal structure may be fabricated according to electron beam melting (EBM) and/or laser engineered net shaping (LENS). For example, with EBM, metallic layers (comprising one or more of the biomaterials, alloys, and substrates disclosed herein) may be coated (layer by layer) on an open cell substrate using an electron beam in a vacuum. Similarly, with LENS, metallic powder (such as a titanium powder, for example) may be deposited and coated on an open cell substrate by creating a molten pool (from a metallic powder) using a focused, high-powered laser beam.

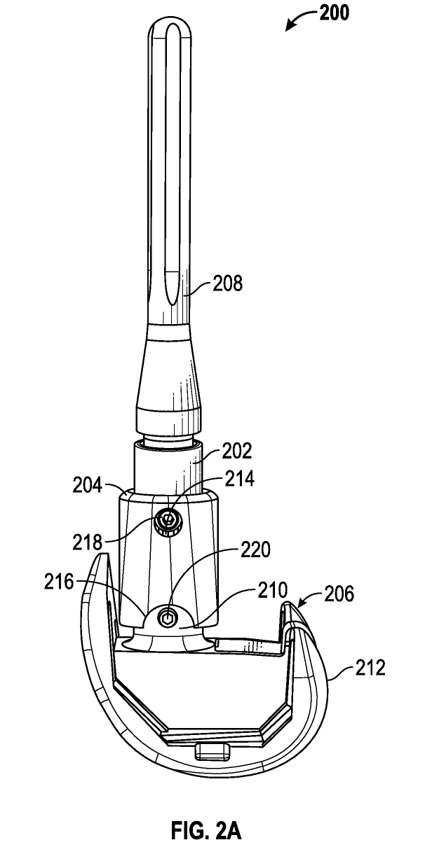

FIGS. 2A and 2B depict elevation and section views, respectively of an example distal femoral prosthesis 200 including adaptor 202 in accordance with this disclosure. As noted above, the use of bone augments in prosthetic systems can complicate modularity, as surgeons may wish to mix and match augments and femoral and tibial prosthetic components from different surgical kits and from different manufacturers. As such, examples according to this disclosure are directed to an adaptor, e.g., adaptor 202, which can be inseparably coupled to a bone augment and which is configured to be connected to an epiphyseal replacement portion and to a diaphyseal anchoring portion of a modular joint replacement prosthesis.

In the example of FIGS. 2A and 2B, adaptor 202 is inseparably coupled to bone augment 204. Bone augments and adaptors in accordance with this disclosure are described as being “inseparably” coupled to one another. In this disclosure, an adaptor and augment are inseparably coupled in the sense that the two components are generally used during a surgical procedure as a single component, where the adaptor and augment are not adjustable relative to one another and where the two components are not disconnected. Thus, while it may be possible to physically separate the augment and adaptor, the two components are configured to be inseparable and used as a single component during a joint repair or replacement procedure. The adaptor can be configured to be a generic adaptor. For example, the two ends of the adaptor can be configured to be connected to different epiphyseal replacement portions and different diaphyseal anchoring portions, respectively, of a modular joint replacement prosthesis. In this manner, a bone augment including an inseparable generic adaptor can be mixed and matched with different prosthetic components, e.g. femoral or tibial, including, e.g., from different surgical kits and from different manufacturers.

Referring to FIGS. 2A, distal femoral prosthesis 200 includes epiphyseal replacement portion 206 and intramedullary stem 208. Epiphyseal replacement portion 206 includes shaft 210 and condylar portion 212 with a medial and a lateral condyle. Bone augment 204 includes thru hole 214 and slot 216. Thru hole 214 can be a threaded hole configured to receive set screw 218. Slot 216 can be configured to provide clearance to insert and tighten set screw 220, which is received in a threaded hole in shaft 210 of epiphyseal replacement portion 206. The proximal end of adaptor 202 is coupled to intramedullary stem 208. The distal end of adaptor 202 is coupled to epiphyseal replacement portion 206. The interconnection between adaptor 202 and augment 204 and adaptor 202 and epiphyseal replacement portion 206 and stem 208 is illustrated in greater detail in FIG. 2B.

Referring to FIG. 2B, adaptor 202 is inseparably coupled to augment 204. In the example of FIG. 2B, adaptor 202 and augment 204 are coupled by a taper-lock, which is also referred to as a self-locking taper. Adaptor 202 interlocks with augment 204 by means of a male taper formed on outer surface 222 of adaptor 202 mated with a complementary female taper formed on inner surface 224 of augment 204.

Adaptor 202 is connected to epiphyseal replacement portion 206 and intramedullary stem 208. For example, distal end of adaptor 202 is connected to epiphyseal replacement portion 206 and proximal end of adaptor 202 is connected to stem 208. Distal end of adaptor 202 includes a shaft that defines outer surface 226. Inscribed in outer surface 226 is channel 228. Shaft 210 of epiphyseal replacement portion 206 includes a bore that defines inner surface 230. Outer surface 226 of adaptor 202 defines a male taper that is configured to be received by and interlocked with a female taper defined by inner surface 230 of the bore of shaft 210.

Proximal end of adaptor 202 includes a bore that defines inner surface 232. The distal end of intramedullary stem 208 includes tapered portion 234. Inscribed in tapered portion 234 of stem 208 is channel 228. Inner surface 332 of adaptor 202 defines a female taper that is configured to receive and interlocked with a male taper defined by tapered portion 234 of stem 208.

To ensure a secure fit between adaptor 202 and augment 204 and between adaptor and epiphyseal replacement portion 206 and stem 208, the taper angle can be chosen to be within the range of self-locking tapers. In one example, the angle, t, of the male taper of adaptor 202 relative to the female taper of augment 204 is in a range from about 1 to about 35 arcminutes, or, from about 1/60 degrees to about 35/60 degrees. In one example, a total included taper angle (both sides of the taper-lock) of adaptor 202 and any of bone augment 204, epiphyseal replacement portion 206, and stem 208 in the range of from about 6 degrees to about 19 degrees can be employed. Other particular taper configurations can also be employed to inseparably couple adaptor 202 and augment 204 and to connect adaptor 202 and epiphyseal replacement portion 206 and stem 208.

In the example of FIGS. 2A and 2B, adaptor 202 is inseparably coupled to augment 204 and connected to epiphyseal replacement portion 206 and stem 208 via a taper-lock. As noted, however, other mechanisms may be employed to connect adaptor 202, augment 204, epiphyseal replacement portion 206, and/or stem 208. For example, adaptor 202 can be inseparably coupled to augment 204 using a press or interference fit. In one example, adaptor 202 is interference fit with augment 204. For example, adaptor 202 and augment 204 can be interference fit to one another using thermal expansion of one or both of the components. This type of coupling may also be referred to as a shrink-fit. In one example employing an interference fit, the phenomenon of thermal expansion is employed to couple adaptor 202 and augment 204 by heating or cooling one of the components before assembly and then allowing the heated/cooled component to return to an ambient temperature after assembly.

In some examples, the taper-lock between adaptor 202 and augment 204 and between adaptor 202 and epiphyseal replacement portion 206 and/or stem 208 can be augmented by surface features on the male and/or female taper. For example, outer surface 222 that forms the male taper of adaptor 202 may include surface features that enhance the interlock between adaptor 202 and augment 204. In one example, complementary inner surface 224 of augment 204 may include a shallow female thread and outer surface 222 of adaptor 202 may be texturized such that the roughness of surface 222 is configured to engage the female thread inscribed in inner surface 224 of augment 204.

Additionally, in some cases the taper-lock, press fit, and/or interference fit between adaptor 202 and augment 204 can be augmented by additional coupling mechanisms. In one example, adaptor 202 and augment 204 are taper-locked to one another and subsequently welded or adhered to one another to complete the coupling of the two components.

The taper-lock described above between adaptor 202 and epiphyseal replacement portion 206 and stem 208 may not be configured to provide a permanent connection between components. In some cases, the taper-lock may be configured to allow a surgeon to connect adaptor 202 and epiphyseal replacement portion 206 and stem 208 and position the components relative to one another. However, the taper-lock may not be configured to be strong or durable enough to provide a permanent connection between the components.

In some examples, therefore, the connections between adaptor 202 and epiphyseal replacement portion 206, and between adaptor 202 and stem 208 can be achieved or augmented by set screws 218 and 220, respectively. For example, set screw 218 is threaded to augment 204 and is driven through a hole in adaptor 202 into channel 236 in tapered portion 234 of stem 208 to secure the adaptor and the stem together. The set screw 218 can function to inhibit relative axial movement between adaptor 202 and stem 208, as well as inhibiting relative rotation between the two components. Similarly, set screw 220 is threaded to shaft 210 of epiphyseal replacement portion 206 and is driven through shaft 210 into channel 228 in adaptor 202. The set screw 220 can function to inhibit relative axial movement between adaptor 202 and epiphyseal replacement portion 206, as well as inhibiting relative rotation between the two components.

Adaptor 202 can be fabricated from a variety of biologically compatible materials, e.g., including the materials described above with reference to adaptor 116. For example, adaptor 202 can be fabricated from a cobalt-chromium alloy.

Augment 204 can be fabricated from bone ingrowth materials such as those described above with reference to bone augments 112 and 124. For example, augment 204 can be fabricated from one or multiple pieces of highly porous biomaterial with a porosity as low as 55%, 65%, or 75% or as high as 80%, 85%, or 90%. An example of such a material is produced using Trabecular Metal™ Technology generally available from Zimmer, Inc., of Warsaw, Ind.

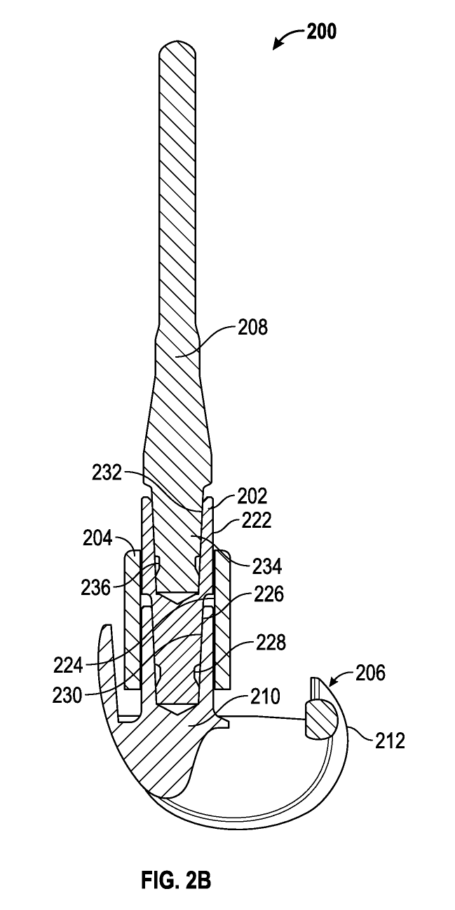

FIG. 3 depicts an example proximal tibial prosthesis 300 including another example adaptor 302 in accordance with this disclosure. In the example of FIG. 3, adaptor 302 is inseparably coupled to augment 304 via bushing 306. Tibial prosthesis includes adaptor 302, augment 304, bushing 306, platform 308, and stem 310.

In some cases, attaching augment 304 to bushing 306, rather than directly to adaptor 302, can assist in situating augment 304 in the more proximal, metaphyseal region of the tibia after the adaptor 302, augment 304, and bushing 306 have been inserted. In some situations, this region is more likely to sustain cavitary damage during revisions. Therefore, bushing 306 can act as a connector between augment 304 and adaptor 302. In some examples, the shape of the bushing 306 is designed to frictionally (press) fit with adaptor 302 with concurrent assembly and weldment of both parts; being adaptable to augment 304, such that adaptor 302, augment 304, and bushing 306 can be eventually permanently attached together; and clear the entire platform 308. After coupling bushing 306 and adaptor 302 the two components effectively become one component. However, bushing 306 and adaptor 302 can be machined separately to simplify the manufacturing process and reduce associated costs.

In one example, proximal tibial prosthesis 300 can includes an epiphyseal replacement portion including tibial platform 308 that is configured to be connected with a tibial bearing (not shown). The tibial bearing mounted to tibial platform 308 forms a concave bearing surface against which the convex condyler surfaces of the patient's femur or a femoral prosthetic are configured to slide. Tibial prosthesis 300 also includes augment 304 and intramedullary stem 310. Bone augment 304 is interposed between platform 308 and intramedullary stem 310. Adaptor 302 is arranged within and coupled to a central cavity of augment 304. The proximal end of adaptor 302 is connected to tibial platform 308 and the distal end is connected to intramedullary stem 310. Intramedullary stem 310 is configured to be inserted within a medullary cavity of the diaphysis of a bone of the patient's knee joint.

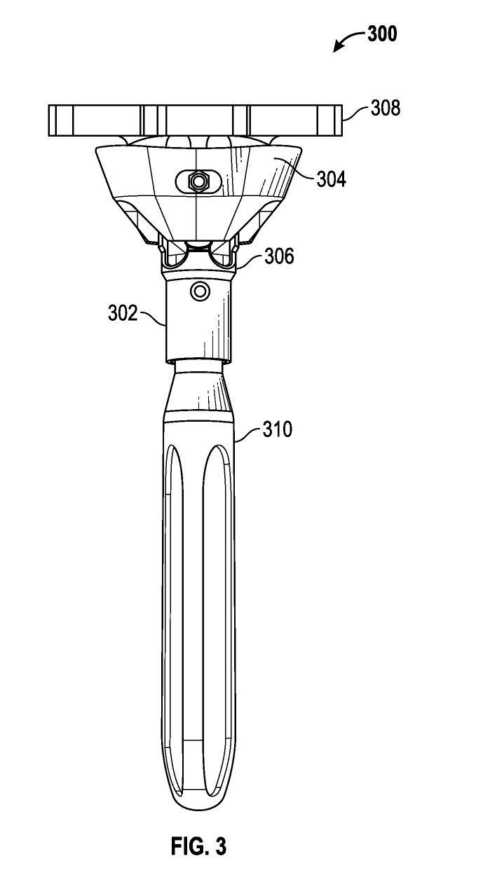

FIGS. 4A and 4B depict adaptor 302, augment 304, and bushing 306 in greater detail. In FIGS. 4A and 4B, adaptor 302 is inseparably coupled to augment 304 via bushing 306. For example, bushing 306 is coupled to adaptor 302 and augment 304 is coupled to bushing 306. The connections between adaptor 302, augment 304, and bushing 306 can be achieved with a variety of mechanisms including those described above with reference to FIGS. 1-2B, e.g., taper-lock, press fit, interference fit, weldment, and the like.

In one example, adaptor 302 and bushing 306 are coupled by a taper-lock. For example, adaptor 302 can interlock with bushing 306 by means of a male taper formed on shoulder 400 of adaptor 302 mated with a complementary female taper formed on inner surface 402 of bushing 306. In another example, shoulder 400 and inner surface 402 can be press or interference fit to one another to couple adaptor 302 to bushing 306.

Adaptor 302 and bushing 306 can be fabricated from a variety of biologically compatible materials, e.g., including the materials described above with reference to adaptor 116. For example, adaptor 302 and bushing 306 can be fabricated from a cobalt-chromium alloy.

Augment 304 can be fabricated from bone ingrowth materials such as those described above with reference to bone augments 112 and 124. For example, augment 304 can be fabricated from one or multiple pieces of highly porous biomaterial with a porosity as low as 55%, 65%, or 75% or as high as 80%, 85%, or 90%. An example of such a material is produced using Trabecular Metal™ Technology generally available from Zimmer, Inc., of Warsaw, Ind.

Bushing 306 can also be connected to augment 304 by a taper-lock. For example, bushing 306 can interlock with augment 304 by means of a male taper formed on outer surface 404 of bushing 306 mated with a complementary female taper formed on inner surface 406 of augment 304. In another example, outer surface 404 and inner surface 406 can be press or interference fit to one another to couple bushing 306 to augment 304.

Adaptor 302 is configured to be connected to tibial platform 308 and intramedullary stem 310. For example, the proximal end of adaptor 302 can be connected to platform 308 and the distal end of adaptor 302 can be connected to stem 310. The proximal end of adaptor 302 includes a shaft that defines outer surface 408. Inscribed in outer surface 408 is channel 410. In one example, platform 308 can include a distally extending protrusion, e.g., a shaft including a bore. The proximal end of adaptor 302 can be configured to be received in the bore of the shaft of platform 308. For example, outer surface 408 of adaptor 302 can define a male taper that is configured to be received by and interlocked with a female taper defined by an inner surface of the bore of shaft extending distally from tibial platform 308.

Proximal end of adaptor 302 includes a bore that defines inner surface 412. In one example, the distal end of intramedullary stem 310 can includes a tapered portion that is configured to with inner surface 412 of adaptor 302. For example, inner surface 412 of adaptor 302 can define a female taper that is configured to receive and interlocked with a male taper defined by the tapered portion of stem 310.

In other examples, other mechanisms may be employed to connect adaptor 302 to platform 308 and/or stem 310. For example, adaptor 302 can be coupled to platform 308 and/or stem 310 using a press or interference fit. In one example, adaptor 302 is interference fit with platform 308 and/or stem 310. For example, adaptor 302 and platform 308 can be interference fit to one another using thermal expansion of one or both of the components. In one example employing an interference fit, the phenomenon of thermal expansion is employed to couple adaptor 302 and platform 308 by heating or cooling one of the components before assembly and then allowing the heated/cooled component to return to an ambient temperature after assembly.

In some examples, the taper-lock between any of adaptor 302, augment 304, bushing 306, platform 308, and/or stem 310 can be augmented by surface features on the male and/or female taper. For example, outer surface 408 that forms the male taper of adaptor 302 may include surface features that enhance the interlock between adaptor 302 and platform 308.

The taper-lock described above between adaptor 302, platform 308, and stem 310 may not be configured to provide a permanent connection between components. In some cases, the taper-lock may be configured to allow a surgeon to connect adaptor 302 and platform 308 and stem 310 and position the components relative to one another. However, the taper-lock may not be configured to be strong or durable enough to provide a permanent connection between the components.

In some examples, therefore, the connections between adaptor 302 and platform 308, and between adaptor 302 and stem 310 can be achieved or augmented by one or more set screws or other appropriate fastening mechanisms. For example, a set screw can be threaded to hole 414 in adaptor 302 into a channel inscribed in the tapered portion of stem 310 to secure the adaptor and the stem together. The set screw can function to inhibit relative axial movement between adaptor 302 and stem 310, as well as inhibiting relative rotation between the two components. Similarly, a set screw can be threaded to a hole in the distally extending shaft of platform 308 and can be driven into channel 410 in adaptor 302. The set screw can function to inhibit relative axial movement between adaptor 302 and tibial platform 308, as well as inhibiting relative rotation between the two components. Hole 416 in augment 304 can be configured to provide clearance for accessing the set screw threaded into platform 308 and configured to engage channel 410.

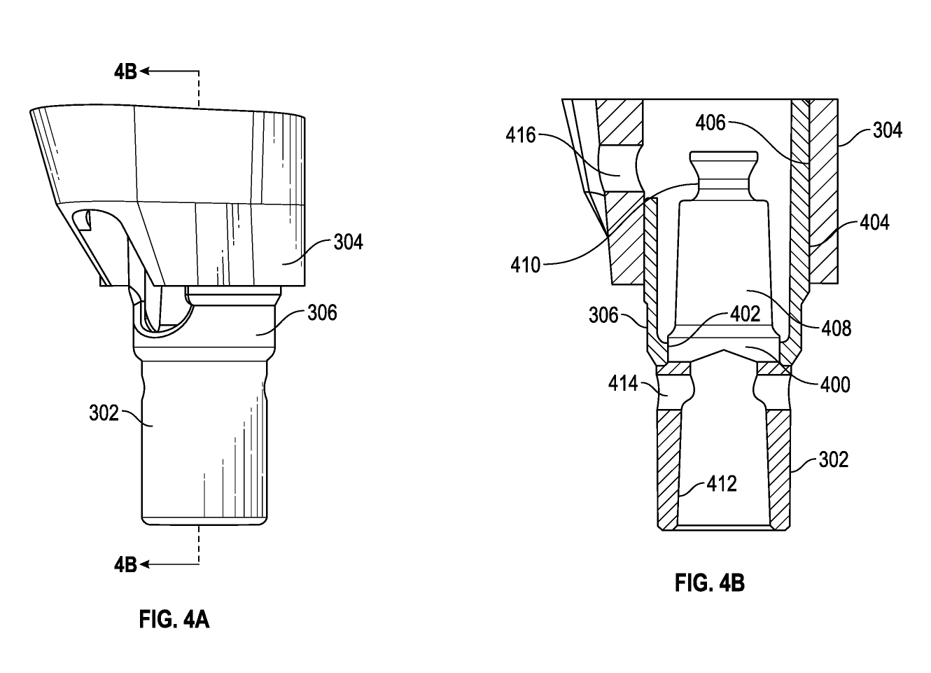

FIG. 5 is a flowchart depicting an example method in accordance with this disclosure. The method of FIG. 5 includes inseparably coupling an adaptor to a bone augment (500), connecting a first end of the adaptor to an epiphyseal component of a prosthetic device (502), and connecting a second end of the adaptor to an intramedullary stem of the prosthetic device (504). In one example, the adaptor and the bone augment are inseparably coupled to one another before a procedure to implant the prosthetic device to partially or completely replace a human joint. The first and second ends of the adaptor can then be connected to the epiphyseal component and the stem, respectively, during the procedure.

For example, the adaptor and augment are inseparably coupled such that the two components are generally used during the surgical procedure as a single component, where the adaptor and augment are not adjustable relative to one another and where the two components are not disconnected. Thus, while it may be possible to physically separate the augment and adaptor, the two components are configured to be inseparable and used as a single component during a joint repair or replacement procedure.

In one example, the adaptor includes a tapered outer surface, which is configured to be received within a cavity of the bone augment. The bone augment can be, e.g., a diaphyseal or metaphyseal femoral augment that augments the cut proximal end of the femur to which the prosthesis is attached. The cavity of the augment includes a tapered inner surface. For example, the femoral augment can include a central bore that includes a tapered profile along a portion or all of the axial length of the bore. The tapered outer surface of the adaptor and the tapered inner surface of the central cavity of the augment are configured to interlock the adaptor and the augment.

The adaptor can be configured to be a generic adaptor. For example, the two ends of the adaptor can be configured to be connected to different epiphyseal replacement portions and different diaphyseal anchoring portions, respectively, of a modular joint replacement prosthesis. During the surgical procedure, the first end of the adaptor is configured to be coupled to an epiphyseal component of the knee repair or replacement prosthesis. For example, the first end of the adaptor can be coupled to a distal femoral component, which includes medial and lateral condyles. The second end of the adaptor is configured to be coupled to an intramedullary stem of the knee repair or replacement prosthesis, which is configured to be affixed within the medullary cavity of the femur.

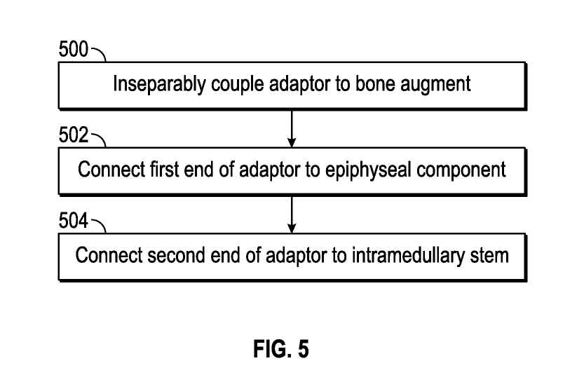

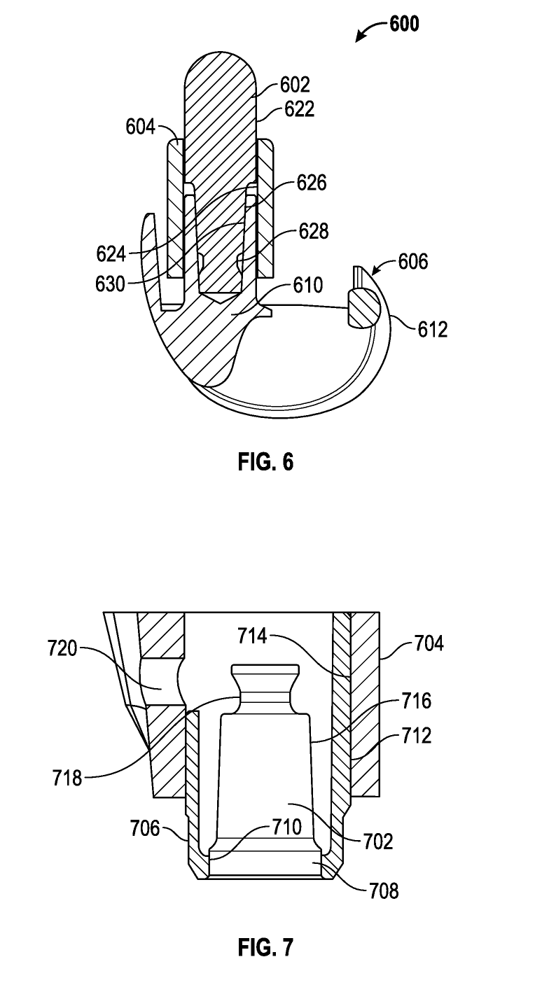

Due to a number of factors including circumstances encountered during surgery and individual patient anatomy, it may not be possible or appropriate to employ an intramedullary stem component that is inserted into and affixed within the intramedullary canal of a patient's bone in a joint repair/replacement procedure. FIGS. 6 and 7 depict two examples of representative devices in accordance with this disclosure that can be employed in such circumstances, or, as appropriate, in other circumstances.

FIG. 6 depicts a section view of a distal femoral prosthesis 600 including an example adaptor 602 in accordance with this disclosure. The example of FIG. 6 is similar to the example of FIGS. 2A and 2B, except the prosthesis does not include an intramedullary stem. In the example of FIG. 6, adaptor 602 is inseparably coupled to bone augment 604. Distal femoral prosthesis 600 includes epiphyseal replacement portion 606. Epiphyseal replacement portion 606 includes shaft 610 and condylar portion 612 with a medial and a lateral condyle. Bone augment 604 may include a slot and/or thru hole (not shown) similar in structure and function to slot 216 and thru hole 214 of the example of FIGS. 2A and 2B. The distal end of adaptor 602 is coupled to epiphyseal replacement portion 606. The proximal end of adaptor 602 is in the form of a truncated shaft, which extends a relatively short distance past the proximal end of augment 604.

The materials employed for components of distal femoral prosthesis 600 can be similar to those described above with reference to distal femoral prosthesis 200 of FIGS. 2A and 2B. Additionally, the interconnection between adaptor 602 and augment 604 and adaptor 602 and epiphyseal replacement portion 606 can be similar in function and structure as that described above with reference to the example of FIGS. 2A and 2B. For example, adaptor 602 is inseparably coupled to augment 604. In the example of FIG. 6, adaptor 602 and augment 604 are coupled by a taper-lock, which is also referred to as a self-locking taper. Adaptor 602 interlocks with augment 604 by means of a male taper formed on outer surface 622 of adaptor 602 mated with a complementary female taper formed on inner surface 624 of augment 604.

Adaptor 602 is connected to epiphyseal replacement portion 606. For example, distal end of adaptor 602 is connected to epiphyseal replacement portion 606. Distal end of adaptor 602 includes a shaft that defines outer surface 626. Inscribed in outer surface 626 is channel 628, which can be configured, for example, to receive a set screw through holes/slots in augment 604 and shaft 610 of epiphyseal replacement portion 606. Shaft 610 of epiphyseal replacement portion 606 includes a bore that defines inner surface 630. Outer surface 626 of adaptor 602 defines a male taper that is configured to be received by and interlocked with a female taper defined by inner surface 630 of the bore of shaft 610.

As noted above, in the example of FIG. 6, distal femoral prosthesis 600 does not include and adaptor 602 does not connect to an intramedullary stem. Instead, the proximal end of adaptor 602 is in the form of a truncated shaft, which extends a relatively short distance past the proximal end of augment 604. The example of FIG. 6 can be employed in situations in which it may not be possible or appropriate to employ an intramedullary stem component that is inserted into and affixed within the intramedullary canal of a patient's bone in a joint repair/replacement procedure.

FIG. 7 depicts another example adaptor 702 in accordance with this disclosure. The example of FIG. 7 is similar to the example of FIGS. 3-4B, except the tibial prosthesis used with the depicted adaptor 702 and augment 704 does not include and the adaptor 702 is not connected to an intramedullary stem. In the example of FIG. 7, adaptor 702 is inseparably coupled to augment 704 via bushing 706. The tibial prosthesis employed with adaptor 702 and augment 704 can be similar in structure and function to the prosthesis of FIG. 3 without the intramedullary stem.

Adaptor 702 is inseparably coupled to augment 704 via bushing 706. For example, bushing 706 is coupled to adaptor 702 and augment 704 is coupled to bushing 706. The connections between adaptor 702, augment 704, and bushing 706 can be achieved with a variety of mechanisms including those described above with reference to FIGS. 1-2B, e.g., taper-lock, press fit, interference fit, weldment, and the like.

In one example, adaptor 702 and bushing 706 are coupled by a taper-lock. For example, adaptor 702 can interlock with bushing 706 by means of a male taper formed on shoulder 708 of adaptor 702 mated with a complementary female taper formed on inner surface 710 of bushing 706. In another example, shoulder 708 and inner surface 710 can be press or interference fit to one another to couple adaptor 702 to bushing 706.

Bushing 706 can also be connected to augment 704 by a taper-lock. For example, bushing 706 can interlock with augment 704 by means of a male taper formed on outer surface 712 of bushing 706 mated with a complementary female taper formed on inner surface 714 of augment 704. In another example, outer surface 712 and inner surface 714 can be press or interference fit to one another to couple bushing 706 to augment 704.

Adaptor 702 is configured to be connected to a tibial platform similar to platform 308 of the example of FIG. 3. For example, the proximal end of adaptor 702 can be connected to the tibial platform. The distal end of adaptor 702, as noted, is not connected to an intramedullary stem and terminates at the distal end of bushing 706. The proximal end of adaptor 702 includes a shaft that defines outer surface 716. Inscribed in outer surface 716 is channel 718, which can be used, for example, to receive a set screw as described above with reference to the examples of FIGS. 3-4B. In one example, the platform of the tibial prosthesis (e.g., platform 308 from the example of FIG. 3) can include a distally extending protrusion, e.g., a shaft including a bore. The proximal end of adaptor 702 can be configured to be received in the bore of the shaft of platform of the tibial prosthesis. For example, outer surface 716 of adaptor 702 can define a male taper that is configured to be received by and interlocked with a female taper defined by an inner surface of the bore of the shaft extending distally from the tibial platform.

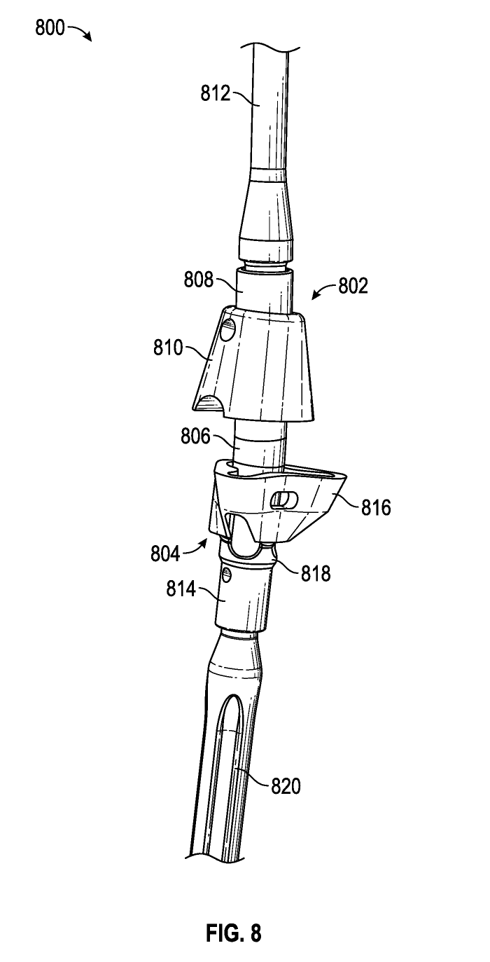

FIG. 8 schematically depicts example modular knee repair or replacement prosthesis 800, which includes bone augment adaptors in accordance with this disclosure. In FIG. 8, prosthesis 800 includes distal femoral prosthesis 802 and tibial prosthesis 804 coupled to one another by a single integral or multiple connected fusion shaft(s) 806. Femoral prosthesis 802 includes adaptor 808 inseparably coupled to augment 810. Adaptor 808 is connected, at the proximal end, to stem 812, and is connected, at the distal end, to fusion shaft 806. Tibial prosthesis includes adaptor 814 inseparably coupled to augment 816 via bushing 818. Adaptor 814 is connected, at the proximal end, to fusion shaft 806, and is connected, at the distal end, to stem 820.

The example of FIG. 8 combines a femoral prosthesis similar to the example of FIGS. 2A and 2B and a tibial prosthesis similar to the example of FIGS. 3-4B, except that knee repair or replacement prosthesis 800 is non-articulating. In other words, prosthesis 800 does not include the articulating components like the condyler epiphyseal distal femoral component or the tibial platform of the examples of FIGS. 1-4B. Example prosthesis 800 may be employed in situations in which it may be necessary to essentially fuse the proximal (femur) and distal (tibia) portions of a patient's leg at the knee. The structure, arrangement, function, and materials of the components of prosthesis 800 can be similar to the examples described above with reference to FIGS. 1-5. However, the distal and proximal ends of adaptors 808 and 814 are respectively connected to fusion shaft 806, instead of being connected to respective articulating epiphyseal components of a femoral and tibial prosthesis.

Various examples have been described. These and other examples are within the scope of the following claims.

1. An prosthetic device configured to partially or completely replace a human joint, the device comprising:
a epiphyseal component configured to be implanted at and replace part or all of the epiphysis of one bone of the human joint;
an intramedullary stem;
a bone augment interposed between the epiphyseal component and the stem; and
an adaptor inseparably coupled to the augment, wherein the adaptor comprises:
a first end coupled to the epiphyseal component; and
a second end coupled to the stem.

2. The prosthetic device of claim 1, wherein the adaptor is received within a central cavity of the augment.

3. The prosthetic device of claim 2, wherein the adaptor comprises a tapered outer surface, wherein the central cavity of the augment comprises a tapered inner surface, and wherein the tapered outer surface of the adaptor and the tapered inner surface of the central cavity of the augment are configured to interlock the adaptor and the augment to one another.

4. The prosthetic device of claim 3, wherein a relative angle between the tapered outer surface of the adaptor and the tapered inner surface of the central cavity of the augment is in a range between about 1 acrminute to about 35 arcminutes.

5. The prosthetic device of claim 1, wherein the first end of the adaptor comprises a male taper configured to interlock with a female taper formed by a cavity in the epiphyseal component.

6. The prosthetic device of claim 5, wherein the male taper of the adaptor comprises a channel, and further comprising a set screw configured to be received in a threaded hole in the augment and engage the channel of the male taper of the adaptor.

7. The prosthetic device of claim 1, wherein the second end of the adaptor comprises a female taper formed by a cavity in the adaptor, wherein the female taper is configured to interlock with a male taper formed at one end of the stem.

8. The prosthetic device of claim 7, wherein the male taper of the stem comprises a channel, and further comprising a set screw configured to be received in a threaded hole in the adaptor and engage the channel of the male taper of the stem.

9. The prosthetic device of claim 4, wherein the first and second ends of the adaptor each comprises a female taper formed by a cavity in the adaptor, wherein the female taper of the first end is configured to interlock with a male taper of the epiphyseal component and the female taper of the second end is configured to interlock with a male taper formed at one end of the stem.

10. The prosthetic device of claim 4, wherein the first and second ends of the adaptor each comprises a male taper, wherein the male taper of the first end is configured to interlock with a female taper of the epiphyseal component and the male taper of the second end is configured to interlock with a female taper formed in one end of the stem.

11. The prosthetic device of claim 1, wherein the adaptor is configured to be coupled to a plurality of different epiphyseal component and a plurality of different stems.

12. The prosthetic device of claim 1, wherein the adaptor comprises titanium.

13. The prosthetic device of claim 1, wherein the epiphyseal component comprises at least one of a proximal femoral component, a distal femoral component, a proximal tibial component, and a distal humeral component.

14. The prosthetic device of claim 1, wherein the adaptor is inseparably coupled to the augment by at least one of a press fit and an interference fit.

15. The prosthetic device of claim 1, further comprising a bushing interposed between the augment and the adaptor, and wherein:
the adaptor is inseparably coupled to the bushing; and
the bushing is inseparably coupled to the augment.

16. An apparatus for a modular prosthetic device configured to partially or completely replace a human joint, the apparatus comprising:
a bone augment configured to be interposed between a epiphyseal component and an intramedullary stem, wherein the epiphyseal component is configured to be implanted at and replace part or all of the epiphysis of one bone of the human joint; and
an adaptor inseparably coupled to the augment, wherein the adaptor comprises:
a first end configured to be coupled to the epiphyseal component; and
a second end configured to be coupled to the stem.

17. The apparatus of claim 16, wherein the adaptor is received within a central cavity of the augment.

18. The apparatus of claim 17, wherein the adaptor comprises a tapered outer surface, wherein the central cavity of the augment comprises a tapered inner surface, and wherein the tapered outer surface of the adaptor and the tapered inner surface of the central cavity of the augment are configured to interlock the adaptor and the augment to one another.

19. The apparatus of claim 16, wherein the first end of the adaptor comprises a male taper configured to interlock with a female taper formed by a cavity in the epiphyseal component.

20. The apparatus of claim 16, wherein the second end of the adaptor comprises a female taper formed by a cavity in the adaptor, wherein the female taper is configured to interlock with a male taper formed at one end of the stem.

21. The apparatus of claim 16, further comprising a bushing interposed between the augment and the adaptor, and wherein:
the adaptor is inseparably coupled to the bushing; and
the bushing is inseparably coupled to the augment.

22. A method comprising:
prior to a procedure to implant a prosthetic device including a bone augment to partially or completely replace a human joint, inseparably coupling an adaptor to the augment;
during the procedure:
connecting a first end of the adaptor to an epiphyseal component of the prosthetic device, wherein the epiphyseal component is configured to be implanted at and replace part or all of the epiphysis of one bone of the human joint; and
connecting a second end of the adaptor to an intramedullary stem of the prosthetic device.
